(19) United States
(12) Patent Application Publication
(10) Pub. No.: US 2023/0309974 A1
(43) Pub. Date: Oct. 5, 2023

(54) Endoluminal Biopsy Tool

(71) Applicants: Technische Universiteit Delft, Delft (NL); Universitair Medisch Centrum Utrecht, Utrecht (NL)

(72) Inventors: Aimée Sakes, Delft (NL); Paulus Breedveld, Delft (NL); Froukje Ellen Euwe, Utrecht (NL)

(21) Appl. No.: 18/331,000

(22) Filed: Jun. 7, 2023

Related U.S. Application Data
(63) Continuation of application No. PCT/NL2021/050731, filed on Dec. 2, 2021.

(30) Foreign Application Priority Data
Dec. 22, 2020 (NL) 2027205

(51) Int. Cl. A61B 10/02; A61B 10/04; A61B 10/00

(52) U.S. Cl. CPC A61B 10/0275; A61B 10/0041; A61B 10/04; A61B 2010/045

(57) ABSTRACT

An endoluminal biopsy tool suitable for use in mammary ductoscopy. The tool includes an outer tube and an instrument in the outer tube such that the outer tube and the instrument are movable with respect to each other for taking the biopsy. The outer tube includes a tongue extending from a distal portion of the outer tube. The instrument includes an inner tube movably fitting inside the outer tube. The inner tube includes a wall that is provided with a cut out which is closable with the tongue.

### CROSS-REFERENCE TO RELATED APPLICATIONS

This application is a continuation of International Patent Application No. PCT/NL2021/050731, titled “An Endoluminal Biopsy Tool”, filed on Dec. 2, 2021, which claims priority to and the benefit of Netherlands Patent Application No. 2027205, titled “An Endoluminal Biopsy Tool”, filed on Dec. 22, 2020, and the specification and claims thereof are incorporated herein by reference.

### BACKGROUND OF THE INVENTION

### Field of the Invention

The invention relates to an endoluminal biopsy tool, which is amongst other applications particularly suitable for use in mammary ductoscopy, said tool comprising an outer tube, and an instrument provided in the outer tube, wherein the outer tube and the instrument are movable with respect to each other for taking the biopsy. The term ‘endoluminal’ with reference to the ‘biopsy tool’ expresses that the biopsy tool to which the invention relates has very limited cross-sectional dimensions which makes it suitable for use in regions of a patient’s body wherein the tool must navigate through very narrow ducts or channels, such as in ductoscopy. The invention should however not be understood as being limited to a biopsy tool for use only in ductoscopy. Possible other applications are:
Lacrimal duct endoscopy
Salivary duct endoscopy
Pancreatic duct endoscopy
Eustachian tube endoscopy
Nephron duct endoscopy
Bile duct endoscopy.

### Background Art

For the sake of clarity, it is nevertheless considered expedient that the following description concentrates on the application of the endoluminal biopsy tool of the invention in ductoscopy.

Early detection of breast cancer plays a vital role in increasing the survival rate of women. Current breast cancer diagnosis modalities, such as mammography and echography, play an important role in the primary screening for breast cancer. MRI plays a vital role in primary screening to diagnose lesions, treatment selection, progression monitoring, and in determining cancer recurrence. However, by the time a lump is felt by the patient herself or with currently clinically available diagnostic tools, the lesion has been growing for approximately 8 years and is usually between Ø5-10 mm in size.

A minimally invasive micro-endoscopic technique called mammary ductoscopy is expected to allow for early breast cancer detection. Mammary ductoscopy allows for the visualization of some of the earliest lesions in situ (Ø0.1 mm) long before traditional imaging modalities, such as mammography, ultrasound, and MRI would allow detection. In mammary ductoscopy, a submillimetre fibreoptic micro-endoscope is inserted through one of the ductal openings onto the nipple surface of the breast to get direct access to the ductal epithelium.

US2006/0058703 discloses a tool usable in mammary ductoscopy, which tool comprises in accordance with the preamble:

(a) a substantially cylindrical cannula with a proximal end and a distal end, said cannula having at least one lateral opening, and (b) an endoscope (in connection with the intended application also referred to as ductoscope) which is axially movable inside the cannula. The at least one lateral opening of the cannula has at least in part a cutting region at its area being directed towards the distal end and/or at its area being directed towards the proximal end. In use a tissue sample is brought through the lateral opening into an interior of the cannula, and the tissue sample is separated from the rest of the tissue by moving the endoscope forward across the lateral opening and/or by retracting the endoscope, until the lateral opening is closed. In another embodiment of taking a biopsy the tissue sample is brought through the lateral opening into an interior of the cannula, and the tissue sample is separated from the rest of the tissue by moving the cannula with the lateral opening together with the fixed endoscope forward or backward, thereby manually exerting a gentle pressure against the tissue sample.

A major disadvantage of the tool known from US2006/0058703 is that taking the biopsy requires moving the endoscope or as it is called in this application the ‘ductoscope’, which implies that at the supreme moment of taking the biopsy the sight at actually taking the biopsy is lost.

### BRIEF SUMMARY OF THE INVENTION

It is therefore an object of the invention to provide a tool which, amongst other purposes, is usable in mammary ductoscopy, and which provides the possibility to maintain sight during the process of actually taking the biopsy.

Embodiments of the present invention are directed to an endoluminal biopsy tool with the features of one or more of the appended claims.

In a first aspect and embodiment of the endoluminal biopsy tool of the present invention, which tool comprises an outer tube and an instrument movably provided in the outer tube, the outer tube comprises a tongue extending from a distal portion of the outer tube, and the instrument is an inner tube movably fitting inside the outer tube, wherein the inner tube has a wall that comprises a cut out which is closable with the tongue.

There are several options that the tongue can close off the cut out. The cut out can for instance be closable by a translational advancing movement of the outer tube with respect to the inner tube.

By the advancing movement of the outer tube with respect to the inner tube the biopsy is taken from the tissue of interest. In that situation it is preferred that the tongue has a forward cutting edge.

Another option is that the cut out is closable by a rotational movement of the outer tube and the inner tube with respect to each other. Then with the rotational movement the biopsy is taken from the tissue of interest. The ease of taking the biopsy is then promoted by arranging that the tongue has cutting side edges.

In a second aspect and embodiment of the present invention the outer tube and the inner tube are translational and/or rotationally movable with respect to each other, the inner tube comprises a bevelled distal portion, and the tongue comprises a preferential position wherein the tongue closes off the bevelled distal portion of the inner tube when the inner tube is placed in a partially retracted position with reference to the outer tube. The benefit of this feature is that by rotating or moving the outer tube forward and keeping the inner tube stationary, the tongue can be brought up to the point that it closes off the bevelled distal portion of the inner tube. During the rotational or forward movement of the tongue it can separate the biopsy from the tissue of interest and assist in moving the biopsy into the inner tube.

The operation of the tongue for closing off the distal portion of the inner tube and for taking the biopsy, is supported by the feature that the tongue has a preferential position wherein the tongue protrudes obliquely towards the inner tube when the tongue is unloaded.

Suitably therefore the tongue is resiliently mounted in an oblique position on the distal portion of the outer tube.

The first embodiment and the second embodiment can be applied independently and separately from each other. When the first embodiment and the second embodiment are combined it is preferable that the cut out is closable with the tongue when the inner tube extends beyond the distal portion of the outer tube, wherein the tongue is loaded by and engages the inner tube.

Suitably when the tongue is loaded by the inner tube, the tongue assumes an essentially straight orientation with reference to the outer tube.

In one embodiment the outer tube is embodied with a further cut out, having dimensions that are substantially the same as the dimensions of the cut out in the inner tube. The further cut out of this embodiment can effectively be used for cutting loose the biopsy tissue, in particular when the further cut out has cutting side edges.

The further cut out can be realized in different ways. The further cut out can be similar in shape to the cut out provided in the inner tube. It is however also possible that the further cut out extends through and is open at the distal portion of the outer tube.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 shows a schematic of current use of a ductoscope in conjunction with other features;

FIGS. 2, 3 and 4 are illustrations from various side views showing aspects of an endoluminal biopsy tool according to an embodiment of the present invention;

FIGS. 5 and 6 are illustrations from various side views showing another endoluminal biopsy tool according to an embodiment of the invention; and

FIGS. 7 and 8 are illustrations from various side views showing another endoluminal biopsy tool according to an embodiment of the present invention.

### DETAILED DESCRIPTION OF THE INVENTION

Whenever in the figures the same reference numerals are applied, these numerals refer to the same parts.

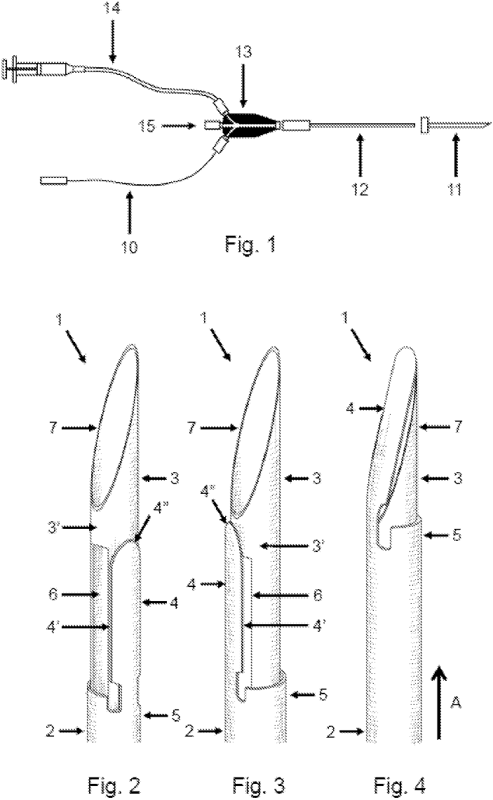

For a proper understanding of how the endoluminal biopsy tool may be used, FIG. 1 shows a schematic of current use of a ductoscope 10 in conjunction with a lumen expander 11, a cannula 12 and a specialized handle 13 that contains 2 or 3 lumens; one lumen for the ductoscope 10, one irrigation lumen 14 (in order to prevent the breast milk ducts from collapsing), and one lumen for an additional tool or treatment modality 15, such as insufflation, ductal lavage, and possible therapeutic interventions. The two main functions of the lumen expander 11 are 1) to provide access to the breast milk ducts via the nipple surface and 2) to extend the milk duct lumen. The cannula 12 provides a conduit for the ductoscope 10, a therapeutic tool 15, and irrigation 14. Irrigation is used to gently dilate the milk duct of interest using saline and allow for advancement of the ductoscope 10 into the duct until further advancement is no longer possible due to the size of the cannula (about Ø1.15-1.4 mm). The lumen of the lumen expander 11 for the additional tool or treatment modality 15 is used for introduction of the endoluminal biopsy tool 1 of the invention into the breast under examination.

FIGS. 2, 3 and 4 show the endoluminal biopsy tool 1 according to an embodiment of the present invention. All figures show that the tool 1 comprises an outer tube 2 and an inner tube 3.

FIG. 2 and FIG. 3 show aspects of a first embodiment of the biopsy tool 1 of the invention, wherein the outer tube 2 is provided with a tongue 4 extending from a distal portion 5 of the outer tube 2, and the inner tube 3 movably fits inside the outer tube 2. It is essential in this first embodiment that the inner tube 3 has a wall 3′ that is provided with a cut out 6 which is closable with the tongue 4. The cut out 6 can for instance be closed by a translational advancing movement of the outer tube 2 with respect to the inner tube 3. For that purpose, it is preferred that the tongue 4 has a forward cutting edge 4″. It is also possible to close off the cut out 6 by a rotational movement of the outer tube 2 and the inner tube 3 with respect to each other. For that situation it is preferred that the tongue 4 has cutting side edges 4′.

Other features that are not necessarily present in the first embodiment and that relate to a second embodiment are also shown in FIGS. 2 and 3. These other features will be referred to hereinafter predominantly with reference to FIG. 4.

In the second embodiment it is essential that the outer tube 2 and the inner tube 3 are translationally and/or rotationally movable with respect to each other. FIG. 4 shows that in this second embodiment the inner tube 3 has a bevelled distal portion 7. The fact that this is also shown in FIGS. 2 and 3 depicts that the first and the second embodiment may be combined.

FIG. 4 further shows that the tongue 4 is provided with a preferential position wherein the tongue 4 closes off the bevelled distal portion 7 of the inner tube 3 when the inner tube 3 is placed in a partially retracted position with reference to the outer tube 2. This can be effectuated by a rotational movement of the outer tube 2 and the inner tube 3 with reference to each other, or by moving the outer tube 2 forward (as symbolized by arrow A) and keeping the inner tube 3 stationary. The latter movement causes that the tongue 4 likewise moves forward up to the point that it can close off the bevelled distal portion 7 of the inner tube 3. This is the situation shown in FIG. 4. During the forward movement of the tongue 4 it can separate the biopsy from the tissue of interest and assist in moving the biopsy into the inner tube 3.

One thing and another is preferably realized in that the tongue is movable between two positions, one position being preferable wherein the tongue 4 protrudes obliquely towards the inner tube 3 when the tongue 4 is unloaded. This is shown in FIG. 4. This can be suitably arranged by the feature that the tongue 4 is resiliently mounted in an oblique position on the distal portion 5 of the outer tube 2.

The other position of the tongue 4 comes in play when the tongue 4 is loaded by and engages the inner tube 3, wherein the tongue 4 assumes an essentially straight orientation with reference to the outer tube 2. This is shown in FIGS. 2 and 3. Since these figures also show features of the first embodiment, the loaded position of the tongue 4 can then be used, as the figures show, to close off the cut out 6 with the tongue 4. It is then required that the inner tube 3 extends beyond the distal portion 5 of the outer tube 2.

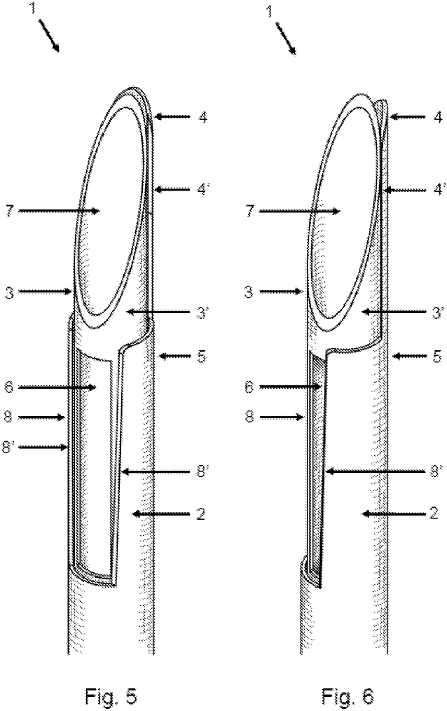

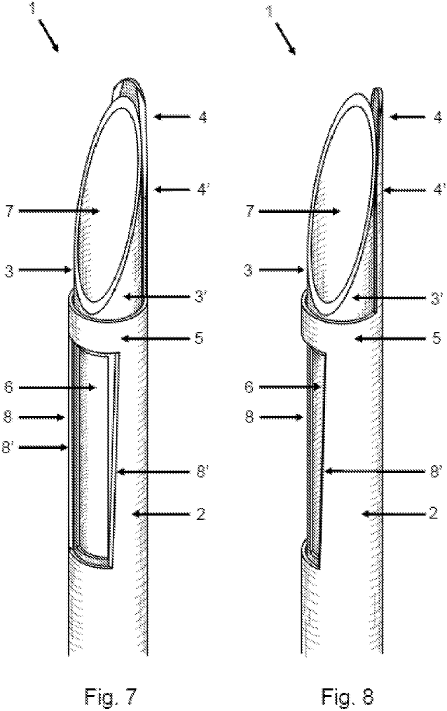

In the two embodiments shown in FIGS. 5 and 6, and FIGS. 7 and 8 respectively, in addition to the features discussed above with reference to the embodiment of FIGS. 2, 3, and 4, the outer tube 2 is embodied with a further cut out 8, having dimensions that are substantially the same as the dimensions of the cut out 6 in the inner tube 3. This further cut out 8 also has a cutting functionality for the biopsy tissue. This is beneficially achieved in that the further cut out 8 has cutting side edges 8′. Rotation of the outer tube 2 with reference to the inner tube 3 when the cut out 6 in the inner tube 3 and the further cut out 8 in the outer tube 2 are overlaying with each other, results in scissor like cutting of tissue that is received in the cut outs 6, 8. The way this operates is clear from comparing FIG. 5 with FIG. 6 and from comparing FIG. 7 with FIG. 8. The difference between the two embodiments shown in FIGS. 5 and 6, and FIGS. 7 and 8 respectively is that in FIGS. 5 and 6 the further cut out 8 extends through and is open at the distal portion 5 of the outer tube 2. In the embodiment of FIGS. 7 and 8 the distal portion 5 of the outer tube 2 is closed in itself.

Although the invention has been discussed in the foregoing with reference to certain examples of the biopsy tool of the invention, the invention is not restricted to what is described, which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another.

What is claimed is:

1. An endoluminal biopsy tool, the tool comprising an outer tube and an instrument in the outer tube, wherein the outer tube and the instrument are movable with respect to each other for taking the biopsy, wherein:
the outer tube comprises a tongue extending from a distal portion of the outer tube;
the instrument is an inner tube movably fitting inside the outer tube; and
the inner tube comprises a wall comprising a cut out which is closable with the tongue.

2. The endoluminal biopsy tool of claim 1, wherein the cut out is closable by a translational advancing movement of the outer tube with respect to the inner tube.

3. The endoluminal biopsy tool of claim 1, wherein the tongue has a forward cutting edge.

4. The endoluminal biopsy tool of claim 1, wherein the cut out is closable by a rotational movement of the outer tube and the inner tube with respect to each other.

5. The endoluminal biopsy tool of claim 1, wherein the tongue comprises cutting side edges.

6. The endoluminal biopsy tool according to claim 1 wherein:
the outer tube and the inner tube are translational and/or rotationally movable with respect to each other;
the inner tube comprises a bevelled distal portion; and
the tongue comprises a preferential position wherein the tongue closes off the bevelled distal portion of the inner tube when the inner tube is placed in a partially retracted position with reference to the outer tube.

7. The endoluminal biopsy tool of claim 6, wherein the tongue has a preferential position in which the tongue protrudes obliquely towards the inner tube when the tongue is unloaded.

8. The endoluminal biopsy tool of claim 1, wherein the tongue is resiliently mounted in an oblique position on the distal portion of the outer tube.

9. The endoluminal biopsy tool of claim 1, wherein the cut out is closable with the tongue when the inner tube extends beyond the distal portion of the outer tube, and the tongue is loaded by and engages the inner tube.

10. The endoluminal biopsy tool of claim 9, wherein when the tongue is loaded by the inner tube, the tongue assumes an essentially straight orientation with reference to the outer tube.

11. The endoluminal biopsy tool of claim 1, wherein the outer tube comprises a further cut out, the further cut out comprising dimensions that are substantially the same as the dimensions of the cut out in the inner tube.

12. The endoluminal biopsy tool of claim 11, wherein the further cut out comprises cutting side edges.

13. The endoluminal biopsy tool of claim 11, wherein the further cut out extends through and is open at the distal portion of the outer tube.

14. The endoluminal biopsy tool of claim 12, wherein the further cut out extends through and is open at the distal portion of the outer tube.
